(19) United States
(12) Patent Application Publication
(10) Pub. No.: US 2025/0179513 A1
(43) Pub. Date: Jun. 5, 2025

(54) GENES FOR REGULATING SPIKE-BRANCHED IN WHEAT AND APPLICATION THEREOF

(71) Applicant: Yulin University, Yulin (CN)

(72) Inventors: Wenzhi Nan, Yulin (CN); Tingting Liu, Yulin (CN); Zhonghua Wang, Xianyang (CN); Fugang Wang, Yulin (CN)

(21) Appl. No.: 18/743,117

(22) Filed: Jun. 14, 2024

(30) Foreign Application Priority Data
Dec. 1, 2023 (CN) 202311630268.9

(51) Int. Cl. C12N 15/82

(52) U.S. Cl. CPC C12N 15/8261

(57) ABSTRACT

Genes for regulating spike-branched of a wheat and an application thereof are provided, which relate to the field of crop genetic engineering technologies. The genes for regulating the spike-branched of the wheat include: TtFZP-2A and TtFZP-2B respectively located on Chr-2AS and Chr-2BS for regulating spike-branched traits of a tetraploid wheat, and/or WFZP-2A, WFZP-2B and WFZP-2D located on Chr 2 for regulating spike-branched traits of a hexaploid wheat. The TtFZP-2A and the TtFZP-2B located on the Chr-2AS and the Chr-2BS can regulate the spike-branched traits of the tetraploid wheat, and the WFZP-2A, the WFZP-2B and the WFZP-2D located on the Chr 2 can regulate the spike-branched traits of the hexaploid wheat, which are beneficial for wheat genetic breeding.

### CROSS-REFERENCE TO RELATED APPLICATION

This application claims priority to Chinese Patent Application No. 202311630268.9, filed Dec. 1, 2023, which is herein incorporated by reference in its entirety.

### TECHNICAL FIELD

The disclosure relates to the field of crop genetic engineering technologies, and more particularly to genes for regulating spike-branched of wheat and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24041TBYX-USP1-SL.xml. The XML file is 41,379 bytes; is created on May 10, 2024; and is being submitted electronically via patent center.

### BACKGROUND

Cereal crops are a main source of carbohydrates for humans, and yields of these crops largely depend on their inflorescence architecture, which is determined by meristem types formed by inflorescence meristems (IM). IM of Poaceae Barnhart plants undergoes a recognition process of a branch meristem (BM), followed by a formation of a spikelet meristem (SM). After producing a certain number of branches and spikelets, the BM itself is converted to SM, and the SM produces a floret meristem (FM), finally, the FM produces a floret organ (also referred to as floral organ). In short, a development of inflorescences in the cereal crops generally converts from IM→BM→SM→FM. The branches of the inflorescences in the cereal crops are terminated when the spikelets are obtained, and an overall shape of the inflorescences is limited by a period of formation of the spikelets. Therefore, the formation of the SM is a main determinant of inflorescence morphogenesis. Generally, delaying SM identification can form more branches and lead to larger inflorescences. IM of a normal spike type wheat does not produce any lateral BM, but directly forms SM. However, middle and lower parts of inflorescence of a spike-branched wheat forms the BM (ultimately forming ramified spike) and ectopic SM (ultimately forming four-rowed spike or multirow spike). Since the spike-branched wheat forms multiple spike axes producing multiple spikelets and forms multiple ectopic spikelets, a number of the spikelets is increased, resulting in a significant increase in a number of grains per spike, which is one of three components of yield, and the spike-branched wheat has great potential for yield increase.

Spike-branched traits of a tetraploid wheat are mainly controlled by genes on Chr-2AS and Chr-2BS, and a development of the spike-branched traits of a hexaploid wheat are mainly controlled by genes on Chr-2AS, Chr-2BS and Chr-2DS. The orthologous genes of a FRIZZY PANICLE (FZP) gene of Oryza sativa L. in Chr2 of the wheat encodes AP2/ERF transcription factors, and regulates the formation of the spike-branched traits of the wheat. In the wheat, researched genes include wfzp-2A, wfzp-D, ttbh-a1, ttfzp-2A and ttfzp-2B. However, other researchers have found in their studies on the spike-branched traits of the wheat that plants with the aforementioned gene mutations exhibit four-rowed spike and multirow spike, without producing ramified spike. The spike-branched traits are generally divided into four-rowed spike (FRS), multirow spike (MRS) and ramified spike (RS). Among them, FRS and RS can be inherited stably, but how to form the RS is still unknown.

### SUMMARY

Aiming at the above problems, in the disclosure, WFZP-2A, WFZP-2B and WFZP-2D are found to jointly regulate formation of RS, MRS and FRS traits of a wheat through genetic analysis, 660K chip trait position, comparative transcriptomic analysis, co-segregation analysis of single nucleotide polymorphism-cleaved amplified polymorphic sequence (SNP-CAPS) marker and spike-branched traits and gene editing. Conserved sequences of these genes are mutated to make a normal wheat form the FRS, the MRS and the RS. Specifically, in a tetraploid wheat, a plant with a homozygous single mutation of TtFZP-2A exhibits an FRS trait; a plant with homozygous double mutations of the TtFZP-2A and TtFZP-2B exhibits a RS trait; and a plant with a homozygous mutation of the TtFZP-2A and a heterozygous mutation of the TtFZP-2B exhibits an MRS trait. In a hexaploid wheat, a plant with simultaneous mutations of the WFZP-2A, the WFZP-2B and the WFZP-2D exhibits the RS trait; and a plant with homozygous mutation of WFZP-2A or WFZP-2D exhibits the FRS trait. The disclosure provides an application of conserved sequences of wheat FRIZZY PANICLE (WFZP) for regulating the spike-branched traits.

In order to achieve the above purposes, the disclosure can adopt the following technical solutions.

A first aspect of the disclosure provides genes for regulating spike-branched traits of a wheat, and the genes include at least one of:
(1) TtFZP-2A and TtFZP-2B respectively located on Chr-2AS and Chr-2BS for regulating the spike-branched traits of the tetraploid wheat; and
(2) WFZP-2A, WFZP-2B and WFZP-2D located on Chr 2 for regulating the spike-branched traits of the hexaploid wheat.

A second aspect of the disclosure provides primer pairs, and the primer pairs are used for polymerase chain reaction (PCR) amplification of the genes for regulating the spike-branched traits of the wheat as described above.

A third aspect of the disclosure provides a biomaterial, and the biomaterial includes the genes for regulating the spike-branched traits of the wheat as described above.

A fourth aspect of the disclosure provides an application method of the genes for regulating the spike-branched traits of the wheat, the primer pairs or the biomaterial as described above, and the application method includes:
performing wheat genetic breeding by using the genes for regulating the spike-branched traits of the wheat, the primer pairs or the biomaterial.

A fifth aspect of the disclosure provides a wheat germplasm resource with an edited WFZP gene, and the wheat germplasm resource includes mutant genes leading to formation of the FRS trait and RS trait in wheat.

Beneficial effects of the disclosure at least include the follows. The genes (i.e., the WFZP gene located on Chr2 of the wheat) for regulating the spike-branched traits of the wheat provided by the disclosure can regulate the spike-branched traits (including the RS trait, the MRS trait and the FRS trait) of the tetraploid wheat and the hexaploid wheat, which is beneficial for wheat genetic breeding.

### BRIEF DESCRIPTION OF DRAWINGS

FIG. 1 illustrates a schematic diagram of a number of significantly differentially expressed genes of comparative transcriptomic.

FIG. 2A illustrates a schematic diagram of a number of SNPs per 10 megabases (Mb) on a chromosome 2B (Chr-2B).

FIG. 2B illustrates a schematic diagram of a percentage of SNPs on the Chr-2B between every 10 Mb of mixing pools to SNPs on the Chr-2B between parents.

FIG. 3A illustrates a schematic diagram of a mutation site of nucleic acid of TtFZP-2A, specifically, an arrow indicates the mutation site of the nucleic acid.

FIG. 3B illustrates a schematic diagram of a mutation site of protein of TtFZP-2A, specifically, an arrow indicates the mutation site of the protein.

FIG. 4A illustrates a schematic diagram of SNP sites of a promoter subregion of TtFZP-2B.

FIG. 4B illustrates a continuation diagram of FIG. 4A.

FIG. 5 illustrates a schematic diagram of a TtFZP-2A genotype in an individual plant of tetraploid F2 hybrid progenies.

FIG. 6 illustrates a schematic diagram of a TtFZP-2B genotype in the individual plant of the tetraploid F2 hybrid progenies.

FIG. 7A illustrates a schematic diagram of nucleic acid sequences of WFZP genes of a hexaploid wheat.

FIG. 7B illustrates a continuation diagram of FIG. 7A.

FIG. 8 illustrates a schematic diagram of protein sequences of the WFZP genes of the hexaploid wheat.

FIG. 9 illustrates a schematic diagram of spike-branched type of a mutation of the WFZP gene of a “fielder” wheat.

### DETAILED DESCRIPTION OF EMBODIMENTS

The embodiments provided are for better description of the disclosure, but the content of the disclosure is not limited to the embodiments provided. Therefore, those skilled in the art can make non-essential modifications and adjustments to the embodiments according to the above summary, which still belong to a scope of protection of the disclosure.

Terms used in this article are only intended to describe specific embodiments and are not intended to limit the disclosure. As used herein, it should be understood that terms such as “comprising”, “having” and “including” are intended to indicate presence of features, numbers, operations, components, parts, elements, materials or combinations. The terms of the disclosure are disclosed in the specification, and it is not intended to exclude the possibility that one or more other features, numbers, operations, components, parts, elements, materials or combinations thereof may exist or may be added. As used herein, “/” can be interpreted as “and” or “or” as the case may be.

The embodiments of the disclosure provide genes for regulating spike-branched traits of wheat, and the genes include: TtFZP-2A (with the nucleotide sequence shown in SEQ ID NO: 25 and the amino acid sequence shown in SEQ ID NO: 30) and TtFZP-2B (with the nucleotide sequence shown in SEQ ID NO: 29) respectively located on Chr-2AS and Chr-2BS for regulating spike-branched traits of a tetraploid wheat, and/or WFZP-2A (with the nucleotide sequence shown in SEQ ID NO: 26 and the amino acid sequence shown in SEQ ID NO: 31), WFZP-2B (with the nucleotide sequence shown in SEQ ID NO: 27 and the amino acid sequence shown in SEQ ID NO: 32) and WFZP-2D (with the nucleotide sequence shown in SEQ ID NO: 28 and the amino acid sequence shown in SEQ ID NO: 33) located on Chr 2 for regulating spike-branched traits of a hexaploid wheat.

It should be noted that the above genes for regulating the spike-branched traits of the wheat is WHEAT FRIZZY PANICLE (WFZP) genes located on Chr2 of the wheat, which regulates formation of the spike-branched traits (i.e., RS, MRS and FRS) of the wheat. In addition, sequences of TtFZP-2A and WFZP-2A are consistent, and sequences of TtFZP-2B and WFZP-2B are consistent.

The embodiments of the disclosure further provide primer pairs, and the primer pairs are used for PCR amplification of the genes for regulating the spike-branched traits of the wheat as described above.

In some specific embodiments, the above primer pairs include:
WFZP-AF, CTCACTTCACTTCTGCCATGAGCTC, as shown in SEQ ID NO: 1, and
WFZP-AR, AGCGCGTCCGTTTCAGTGGGAGAG, as shown in SEQ ID NO: 2;
WFZP-BF, GCATCACTTCAGTTGTGCCATGAGC, as shown in SEQ ID NO: 3, and
WFZP-BR, GCCGGTGCATTTGCTTCAGTGTGAG, as shown in SEQ ID NO: 4;
WFZP-DF, CACTTCACTTCAGTTCTGCCATGAG, as shown in SEQ ID NO: 5, and
WFZP-DR, GCTGGTGCATTTGTTGTTTCAGTG, as shown in SEQ ID NO: 6;
WFZP-BF2, GCATCACTTCAGTIGTGCCATG, as shown in SEQ ID NO: 7, and
WFZP-BR2, CAATGCCGGTGCATTTGC, as shown in SEQ ID NO: 8;
WFZP-AF2, CCACAGTGCTCTCACTCCTTCTAGC, as shown in SEQ ID NO: 9, and
WFZP-AR2, TAAGAGCAATGCCAGCGCGTC, as shown in SEQ ID NO: 10; and
WFZP-DF2, CCACAGTGCTCTCACTCCTAGCTTC, as shown in SEQ ID NO: 11, and
WFZP-DR2, CTGAGAGCAATGCTGGCTGGTG, as shown in SEQ ID NO: 12.

It should be noted that the WFZP-AF, the WFZP-AF2, the WFZP-AR and the WFZP-AR2 are used to amplify the WFZP-2A. The WFZP-BF, the WFZP-BF2, the WFZP-BR and the WFZP-BR2 are used to amplify the WFZP-2B. The WFZP-DF, the WFZP-DF2, the WFZP-DR and the WFZP-DR2 are used to amplify the WFZP-2D. In these primers, F and R are a pair of upstream and downstream primers, and F2 and R2 are another pair of upstream and downstream primers.

The embodiments of the disclosure further provide a biomaterial, and the biomaterial includes the genes for regulating the spike-branched traits of the wheat as described above.

The embodiments of the disclosure further provide a wheat germplasm resource with edited WFZP gene, and the wheat germplasm resource includes mutant genes of the genes for regulating the spike-branched traits of the wheat as described above. It should be noted that gene editing is performed on the WFZP-2A, the WFZP-2B and the WFZP-2D in a wheat (fielder wheat) genome, and editing materials include genetically stable FRS strains and RS strains, which can be used for genetically modified purposes.

The embodiments of the disclosure further provide an application of the genes for regulating the spike-branched traits of the wheat, the primer pairs or the biomaterial as described above in wheat genetic breeding.

It should be noted that the above genes can be used in wheat genetic breeding. For example, situations of the genes for regulating the spike-branched traits of the wheat can be detected through the primer pairs before breeding, to select a target parent. For another example, the conserved domain of the WFZP genes in the wheat is modified by using a gene editing technology, to achieve regulation of the spike-branched traits of the wheat. Alternatively, the gene editing germplasm resource of applicants can be used to introduce mutated traits, which can be selected based on specific situations.

It should be noted that in the disclosure, it is found that the TtFZP-2A and the TtFZP-2B of a RS wheat have mutations compared with a normal spike wheat through studying a tetraploid wheat with the RS trait. It is found that a plant with homozygous double mutations of the TtFZP-2A and the TtFZP-2B exhibits the RS trait and a plant with a homozygous mutation of the TtFZP-2A and a heterozygous mutation of the TtFZP-2B exhibits the MRS trait through performing a co-segregation analysis of a gene mutation and the traits on a genetic population of the RS wheat and the normal spike wheat, which implies that mutation of the WFZP gene can regulate the spike-branched traits. The TtFZP-2A and the TtFZP-2B are genetic loci of the WFZP gene on wheat chromosomes 2A and 2B, respectively, which are named as TtFZP-2A and TtFZP-2B. Through performing gene editing on the WFZP gene (having three genetic loci, which are located on chromosomes 2A, 2B and 2D, and are named as WFZP-2A, WFZP-2B and WFZP-2D) of a hexaploid normal spike phenotype wheat “fielder”, it is found that the mutation in the WFZP cause wheat to transition from a normal spike trait to a spike-branched trait, and different mutation combinations of the three genetic loci can cause different spike-branched traits. The WFZP-2A, the WFZP-2B and the WFZP-2D represent three completely genes. In the disclosure, a conserved region of the WFZP gene of the hexaploid wheat is genetically edited, and the inventors consider that conserved region sequences of the WFZP-2A, the WFZP-2B and the WFZP-2D genes are crucial for them to exhibit such biological functions, and the spike-branched phenotype of the wheat can be regulated through mutating the conserved regions of them. Therefore, in the disclosure, the mutations of the conserved sequences of the WFZP gene on the chromosomes 2A, 2B and 2D can achieve the purpose of regulating the spike-branched traits whether the wheat is the tetraploid wheat or the hexaploid wheat.

In order to understand the disclosure better, specific embodiments are combined to further describe contents of the disclosure below, but the contents of the disclosure are not limited to the following embodiments.

### Embodiment 1 Genetic Analysis of the Spike-Branched Traits

GAN-A1582 (female parent with normal spike)×GAN-A631 (male parent with RS) and GAN-A1582 (male parent with normal spike)×GAN-A631 (female parent with RS), as well as GAN-A631 (male parent with RS)×DW1 (female parent with normal spike) and GAN-A631 (female parent with RS)×DW1 (male parent with normal spike) are crossed and self-crossed, and the crossed and self-crossed results indicate that the spike-branched traits are not affected by cytoplasmic genes. Spike-branched trait separation of F2 plants of GAN-A1582 (male parent)×GAN-A631 (female parent) and DW1 (male)×GAN-A631 (female) is consistent with a separation ratio controlled by a pair of recessive nuclear genes (as shown in Table 1).

TABLE 1
Separation of spike-branched traits in F2 populations

| | Spike- | | | Chi-square |
|---|---|---|---|---|
| | branched | Normal | | (χ²) |
| Cross combination | trait | spike | Total | analysis (3:1) |
| GAN-A631/GAN-A1582 | 63 | 170 | 233 | 0.41 |
| GAN-A631/DW1 | 55 | 139 | 194 | 0.99 |

In the spike-branched plants, a separation ratio between the plants with RS phenotype and the plants without branching (FRS and MRS) is 1:3 (as shown in Table 2), the separation of the RS trait is consistent with the separation ratio controlled by a pair of recessive nuclear genes. Therefore, formation of the RS trait of the tetraploid wheat GAN-A631 is controlled by two pairs of recessive genes.

TABLE 2
Separation of RS trait in F2 populations

| Cross combination | RS | MRS | FRS | Total | χ² analysis (3:1) |
|---|---|---|---|---|---|
| GAN-A631/GAN-A1582 | 18 | 13 | 32 | 63 | 0.26 |
| GAN-A631/DW1 | 12 | 17 | 26 | 55 | 0.15 |

### Embodiment 2 Mapping of the Spike-Branched Traits

### (1) Gene Mapping Method

A wheat 660 K chip is used to detect SNP markers of an extreme RS trait mixing pool and an extreme FRS trait mixing pool in GAN-A631, GAN-A1582 and F2: 3 plants of GAN-A1582 (male parent)×GAN-A631 (female parent), to thereby excavate the SNP markers with polymorphism between the parents and between the mixing pools. A local basic local alignment search tool (BLAST) is performed on a chip probe sequence with polymorphic SNP and a genome sequence (Triticum_aestivum.TGACv1.dna.toplevel.fa) to obtain physical location information of the polymorphic SNP on reference genome. A Perl script is used to count a number of polymorphic SNPs between the parents on each chromosome and a number of polymorphic SNPs simultaneously existing between the mixing pools and between the parents, and count a number of the polymorphic SNPs per 10 Mb distance on each chromosome. Concentration of the polymorphic SNPs between the parents and the polymorphic SNPs simultaneously existing between the mixing pools and between the parents on each chromosome is analyzed to determine which segments of the parental chromosome were the genetic loci regulating the RS trait and the FRS trait.

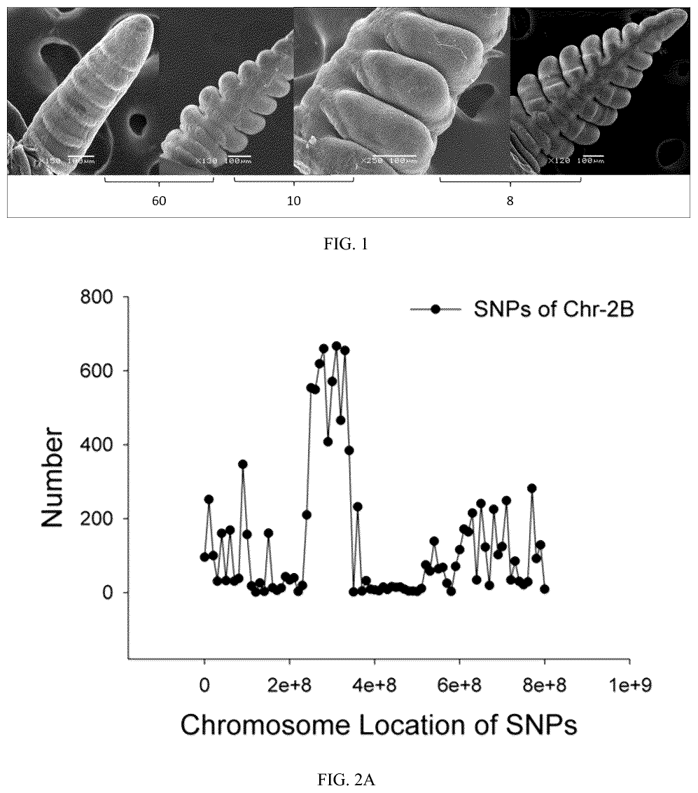

A transcriptome sequencing analysis is performed on a process of GAN-A631 forming BM and SM from IM to perform comparative transcriptome analysis of formation processes of different types of meristems (as shown in FIG. 1). Significant differentially expressed genes obtained from the comparative transcriptomic analysis are compared to the genome sequence (Triticum_aestivum.TGACv1.dna.toplevel.fa) through the BLAST alignment to obtain chromosome location information of the significant differentially expressed genes.

### (2) Gene Mapping Results

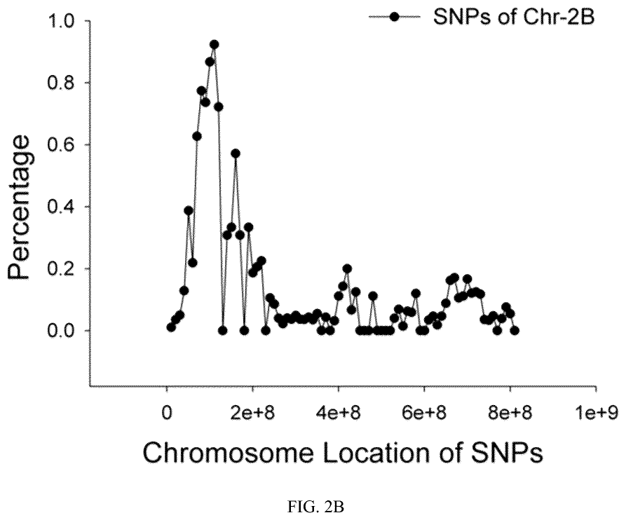

SNP assays in 60˜120 Mb of chromosome 2B were uniquely selected and exhibited distorted segregation between FRS and RS DNA bulks, and 100˜110 Mb of chromosome 2B showed a higher proportion of polymorphic (92.36%), while 250˜330 Mb of chromosome 2B has the highest number of the SNP assays in GAN-A1582 and GAN-A631 parents (as shown in FIG. 2B), which indicates that this chromosome segment is specifically selected during the mixing pool grouping process. Between the parents, a segment with the largest number of polymorphic SNP markers is between 250 Mb to 330 Mb of the Chr-2B (as shown in FIG. 2A), however, the polymorphic SNP in this segment is eliminated as background in the mixing pool grouping process. The same analysis process is used to analyze polymorphic SNPs on other chromosomes, the results show that no significant chromosomal segments are specifically selected during construction of the mixing pool using a cluster separation method for the polymorphic SNPs on other chromosomes. The results indicate that the genetic loci regulating transformation of the traits from FRS to RS may be between 100 Mb to 110 Mb of the Chr-2B.

In addition, the 90 Mb to 110 Mb of the reference genome Chr-2B of Chinese spring wheat contains a total of 116 genes. The disclosure found that 36 genes of the above 116 genes are expressed during an early development process of GAN-A631 growth cone through the transcriptome analysis, and two genes (MSTRG.16311 and MSTRG.15946) are significantly differentially expressed during a transformation process from BM to SM in the RS wheat GAN-A631, and a gene (MSTRG.15495) is significantly differentially expressed during a transformation process from IM to BM in the GAN-A631. The MSTRG.15495 gene is a homologous gene of the WFZP gene on the chromosome 2B, and is named as TtFZP-2B.

### Embodiment 3 Gene Sequence and Promoter of TtFZP-2A and TtFZP-2B Cloned from the Tetraploid Wheat

### (1) Operation Flow of Molecular Biology Experiment of Gene Cloning

a. KOD FX high fidelity DNA polymerase (TOYOBO, KFX-101) is used to perform PCR amplification to obtain amplification sequences shown in Table 3.

TABLE 3
PCR amplification primers

| | | |
|---|---|---|
| Entire length of | wFZP-2BF | GCATCACTTCAGTTGTGCCATGAGC (as |
| TtFZP-2B | | shown in SEQ ID NO: 13) |
| | wFZP-2BR | GCCGGTGCATTTGCTTCAGTGTGAG (as |
| | | shown in SEQ ID NO: 14) |
| Entire length of | wFZP-2AF | CTCACTTCACTTCTGCCATGAGCTC (as |
| TtFZP-2A | | shown in SEQ ID NO: 15) |
| | wFZP-2AR | AGCGCGTCCGTTTCAGTGGGAGAG (as |
| | | shown in SEQ ID NO: 16) |
| Promotor region of | wFZP-2BF | CCCTCTCTCTTCCTCTGTCTACA (as shown |
| TtFZP-2B | | in SEQ ID NO: 17) |
| | wFZP-2BR | ATGGCACAACTGAAGTGATGC (as shown in |
| | | SEQ ID NO: 18) |

The gene amplification system is shown in the following Table 4.

TABLE 4
Gene amplification system

| Total Volume | 50 | microliters (μL) |
|---|---|---|
| 2x PCR buffer for KOD FX | 25 | μL |
| dNTPs (2 millimoles per liter | 10 | μL |
| abbreviated as mM each) | | |
| F (10 micromoles per liter abbreviated | 1.5 | μL |
| as μM) | | |
| R (10 μM) | 1.5 | μL |
| DNA | 200 | nanograms (ng) |
| KOD FX (1.0 unit per microliter | 1 | μL |
| abbreviated as U/μL) | | |
| ddH₂O | Up to 50 | μL |

The PCR amplification program is set as follows: pre-denaturation at 94 Celsius degree (° C.) for 5 minutes (min); denaturation at 98° C. for 10 seconds(s), annealing at melting temperature (Tm)±2° C. for 30 s, extension at 68° C. for 1 kilobase per minute (kb/min), and then 35 cycles for the PCR amplification.
b. A target gene is connected to a clone carrier.
c. A process of transforming the target gene into Escherichia coli DH5a is as follows: a pMD®18-T carrier connected with the target gene is transformed into the Escherichia coli DH5a (CB101, TIANGEN), and the operation process refers to the instructions.
d. A process of Sanger sequencing is as follows: bacterial fluid of the Escherichia coli DH5a transforming the pMD®18-T carrier is coated on a Luria-Bertani (LB) solid culture medium (containing 100 milligrams per liter abbreviated as mg/L ampicillin antibiotic) for culturing at 37° C. for 15 hours (h). A single colony is selected from the LB solid culture medium and is sent to Sangon Biotech (Shanghai) Co., Ltd to sequence, to thereby obtain a sequence of the target gene.

### (2) Gene Cloning Result

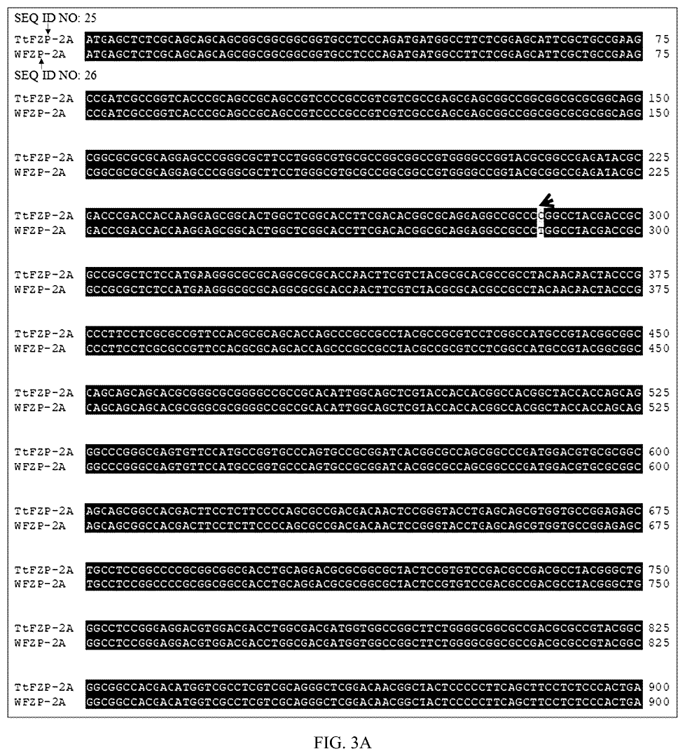

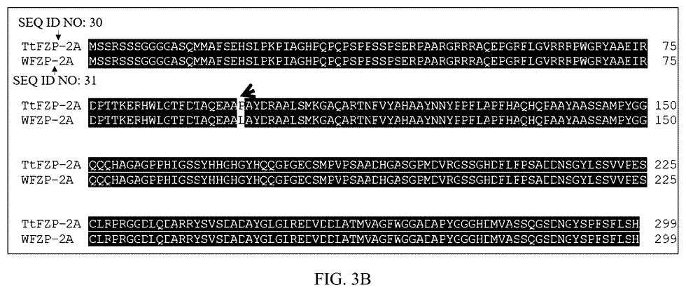

The TtFZP-2A and the TtFZP-2B in the tetraploid RS wheat GAN-A631 and the normal spike wheats GAN-A1582 and DW1 are amplified. The amplification results show that compared to the GAN-A1582 and DW1, GAN-A631 has a single base mutation (T-C) in AP2/ERF domain of the TtFZP-2A gene, which ultimately causes a mutation of amino acid (L-P) (as shown in FIG. 3). The TtFZP-2B gene has no change in three wheat varieties.

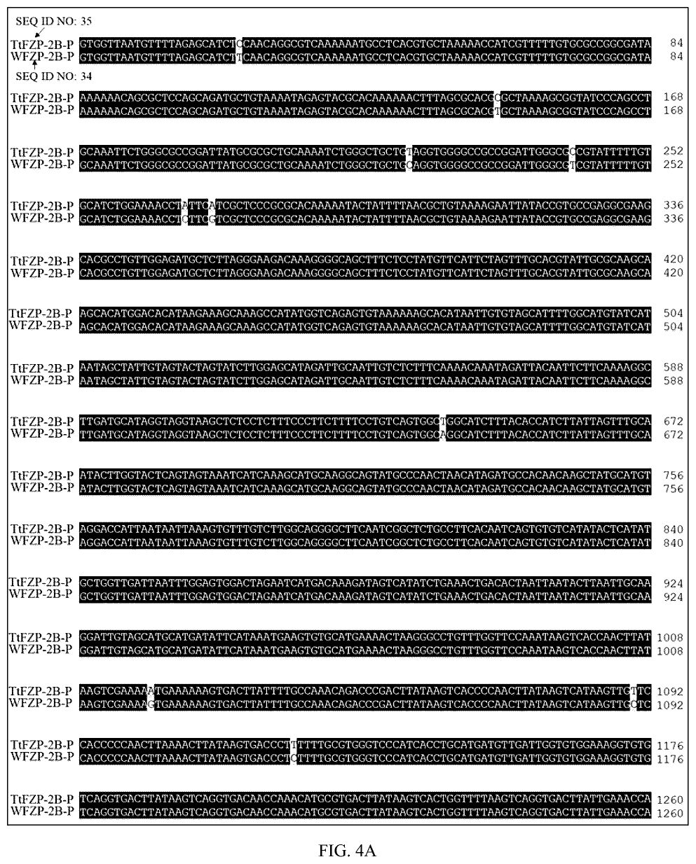

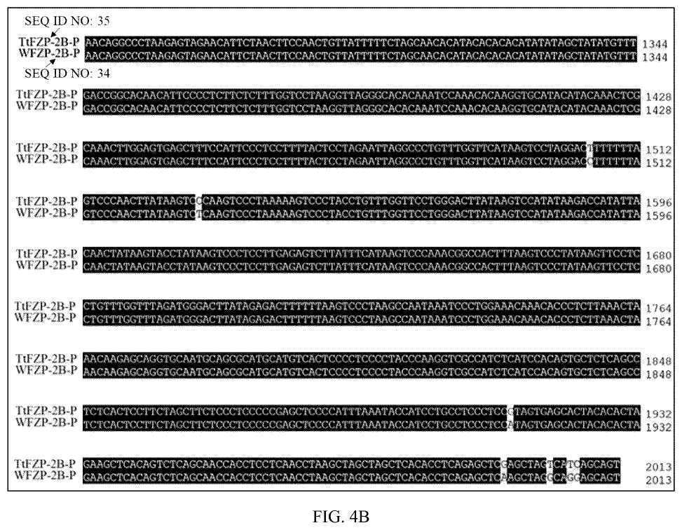

Similarly, the promoter sequences of the TtFZP-2A and the TtFZP-2B are cloned in the above wheat varieties. As shown in FIGS. 4A and 4B, the results show that compared to the GAN-A1582 and DW1, the promoter region of the TtFZP-2B of the GAN-A631 contains multiple SNPs. Specifically, the nucleotide sequence of the WFZP-2B promotor (i.e., WFZP-2B-P) is shown as SEQ ID NO: 34, and the nucleotide sequence of the TtFZP-2B promotor (i.e., TtFZP-2B-P) is shown as SEQ ID NO: 35).

### Embodiment 4 Co-Segregation Analysis of Mutations of Ttfzp-2A and Ttfzp-2B with the Spike-Branched Traits

### (1) Development Method and Co-Segregation Analysis Method of a SNP-CAPS Marker

SNP-CAPS markers FPAcap-F/FPAcap-R are developed according to a single base mutation of ttfzp-2A in the GAN-A631. After PCR amplification, the PCR amplification products are digested by EcoRII enzyme. The PCR amplification product of the normal spike wheat can be digested into product bands of 108 base pairs (bp), 193 bp, and 268 bp, and the PCR amplification product of the RS wheat can be digested into product bands of 193 bp and 376 bp. After digesting the PCR amplification products, sizes of the digested products are determined through polyacrylamide gel to distinguish the type of TtFZP-2A sequence carried by the 240 F2 plants of GAN-A631×GAN-A1582 and GAN-A631×DW1. Similarly, the SNP-CAPS markers FPBcap-F/FPBcap-R are designed by SNP existing in promoter regions of ttfzp-2B gene of the RS wheat and the normal spike wheat. After PCR amplification, the PCR amplification products are digested by SpeI enzyme. The PCR amplification product of the normal spike wheat can be digested into product bands of 142 bp and 342 bp, and the PCR amplification product of the RS wheat cannot be digested by the SpeI enzyme.

A detailed detection process of the SNP-CAPS markers is as follows.
a. PCR Amplification of a target segment

The PCR amplification primers are shown in the following Table 5.

TABLE 5
PCR amplification primers

| | |
|---|---|
| FPAcap-F | 5′-CTCACTTCACTTCTGCCATGAGCTC-3′ (as shown in SEQ ID NO: 19) |
| FPAcap-R | 5′-GGTACGAGCTGCCAATGTGC-3′ (as shown in SEQ ID NO: 20) |
| FPBcap-F | 5′-AGCATCTCCAACAGGCGTG-3′ (as shown in SEQ ID NO: 21) |
| FPBcap-R | 5′-ATGTGTCTTGTGAGCCGTGC-3′ (as shown in SEQ ID NO: 22) |

The PCR amplification system is shown in the following Table 6.

TABLE 6
PCR amplification system

| | | |
|---|---|---|
| Total Volume | 50 | μL |
| 2x PCR buffer for KOD FX | 25 | μL |
| DNTPs (2 mM each) | 10 | μL |
| F (10 μM) | 1.5 | μL |
| R (10 μM) | 1.5 | μL |
| DNA | 200 | ng |
| KOD FX (1.0 U/μL) | 1 | μL |
| ddH₂O | Up to 50 | μL |

The PCR amplification program is set as follows: pre-denaturation at 94° C. for 5 min; denaturation at 98° C. for 10 s, annealing at Tm±2° C. for 30 s, extension at 68° C. for 1 kb/min, and then 35 cycles for the PCR amplification.
b. Digestion of the PCR amplification product by the EcoRII enzyme and the SpeI enzyme.

The digestion system is shown in the following Table 7.

TABLE 7
Digestion system

| | | |
|---|---|---|
| Total Volume | 15 | μL |
| buffer | 1.5 | μL |
| cDNA | 8 | μL |
| EcoRII/SpeI (5.0 U/μL) | 0.8 | μL |
| ddH₂O | Up to 15 | μL |

c. The digested products are detected through polyacrylamide gel electrophoresis, including: 6% polyacrylamide denaturing gel electrophoresis is performed on the digested products. The 6% denaturing polyacrylamide gel electrophoresis includes four steps: gel preparation, spotting, electrophoresis, and silver staining and color development, and the four steps are as follows.

In the gel preparation step, 16 milliliters (mL) acrylamide/bis-acrylamide (Acr-Bis), 9 mL 10×tris-borate-ethylenediaminetetraacetic acid (TBE), 800 μL of 10% ammonium persulfate and 80 μL of N,N,N′,N′-tetramethylethylenediamine (TEMED) are added into 64 mL pure water for stirring evenly to obtain a gel. The gel is poured between two glass plates, the two glass plates after filling up the gel are slanted to form an angle of about 10° with a tabletop, and a comb is inserted into the two glass plates with gel to stand for 40 min.

In the spotting step, the comb is carefully pulled out from the glass plates after the gel has completely solidified, the glass plates are fixed on a vertical electrophoresis tank, and an appropriate amount of 1×TBE buffer solution is added into the glass plates. The PCR amplification products are taken out, and are added with 1 μL loading buffer containing two indicators of bromophenol blue and xylene cyanide to obtain samples, and 2 μL of samples are injected into each spotting well of the glass plates by using a micropipette.

In the electrophoresis step, a voltage of the electrophoresis tank is adjusted to 400 volts (V), and an electrophoresis time is 2.5 h.

In the silver staining and color development step, the glass plates are removed from the electrophoresis tank after completing the electrophoresis, the gel is taken out and is washed in distilled water for twice, about 1 min each time, to obtain washed gel. Then the washed gel is added into 0.1% silver nitrate (AgNO₃) solution, and the AgNO₃ solution added with the washed gel is gently shaken on a table for staining for 25 min to obtain a stained gel. The stained gel is transferred into the distilled water for washing for twice, and then transferred into a color developing solution for color developing, and the color developing results are photographed by a camera and are recorded.

Solution formulas used in the above steps are shown in Table 8.

TABLE 8
Solution formulas
1) 10x TBE

| | | |
|---|---|---|
| Tris | 108 | grams (g) |
| Boric acid | 55 | g |
| Na₂EDTA · 2H₂O | 7.44 | g |

2) 40% (weight/volume abbreviated as L
w/v) acrylamide (Acr/Bis) 1316 m

| | | |
|---|---|---|
| Acrylamide | 500 | g |
| Bis (N, N′-methylene bis acrylamide) | 28 | g |

700 mL water is added, and a volume of
the solution is diluted to 1316 mL
3) 10% (w/v) ammonium persulfate 20 mL
2 g ammonium persulfate is weighed,
and is dissolved in 20 mL pure water

4) staining solution 0.2% (w/v) AgNO₃
1 g AgNO₃ is added into 1 litter (L) water

5) 10x color developing solution

| | | |
|---|---|---|
| NaOH | 150 | g |
| Sodium tetraborate | 1.9 | g |

600 mL pure water is added, and a volume of the solution is diluted to 1 L
When using the color developing solution, it is diluted to a 1x color developing solution, and formaldehyde is added into the 1x color developing solution (960 μL formaldehyde is added into each 400 mL color developing solution).

### (2) Co-Segregation Analysis Result

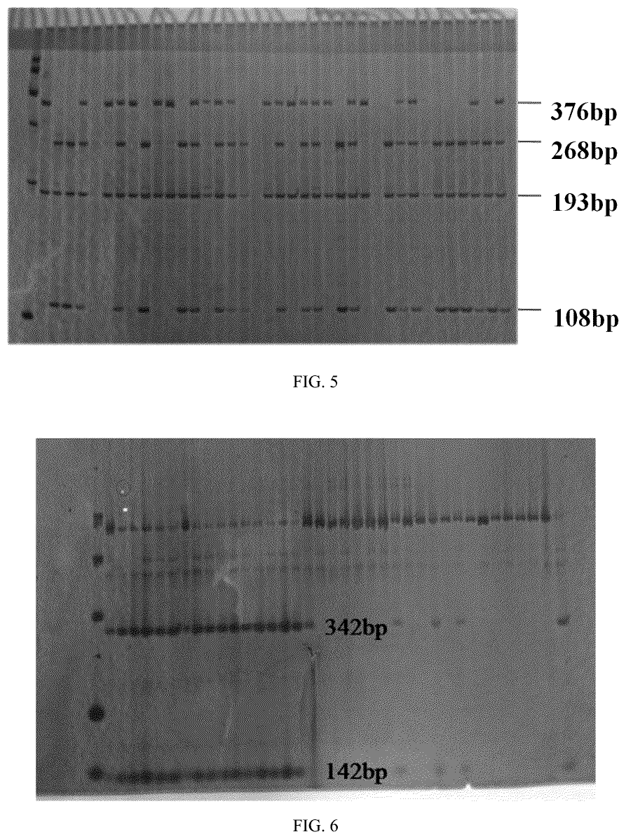

The SNP-CAPS markers based on the mutation of ttfzp-2A are used to perform TtFZP-2A genotyping on F2 individual plants of GAN-A1582 (male parent)×GAN-A631 (female parent) and DW1 (male parent)×GAN-A631 (female parent). Through PCR amplification of target bands and EcoRII enzyme digesting, an individual plant only having 193 bp and 376 bp bands is a TtFZP-2A gene homozygous mutant plant, an individual plant only having 108 bp, 193 bp and 268 bp bands is a TtFZP-2A gene homozygous wildtype plant, and an individual plant having 108 bp, 193 bp, 268 bp and 376 bp bands is a TtFZP-2A gene heterozygous plant (as shown in FIG. 5). The results show that in two F2 separated populations, only a F2 individual plant with the homozygous mutation of the TtFZP-2A will exhibit the spike-branched (FRS, MRS and RS) phenotype.

The SNP-CAPS marker based on a promoter region mutation of ttfzp-2B is used to perform TtFZP-2B genotyping on 150 individual plants in the FRS phenotype DNA mixing pool and 150 individual plants in the RS phenotype DNA mixing pool, respectively. Meanwhile, the TtFZP-2B genotyping is performed on 120 individual plants of each F2 population of GAN-A1582 (male parent)×GAN-A631 (female parent) and DW1 (male parent)×GAN-A631 (female parent), the results show that the FRS individual plants can be digested to 142 bp and 342 bp bands, the RS individual plants cannot be digested by the speI enzyme, and the MRS individual plants often exhibit heterozygous band patterns (as shown in FIG. 6).

### Embodiment 5 WFZP Gene Editing

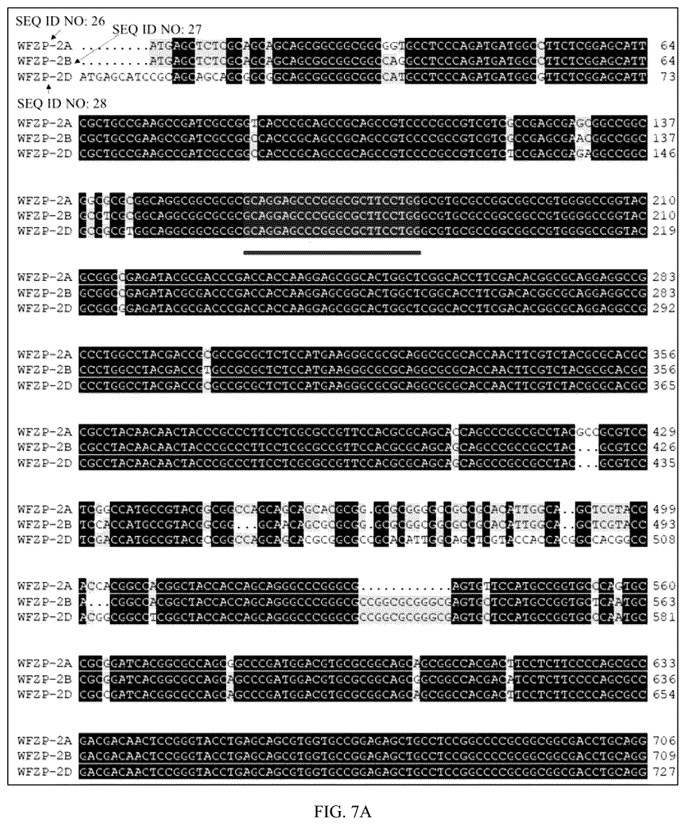

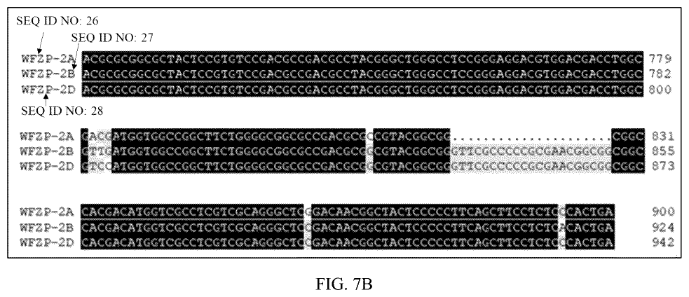

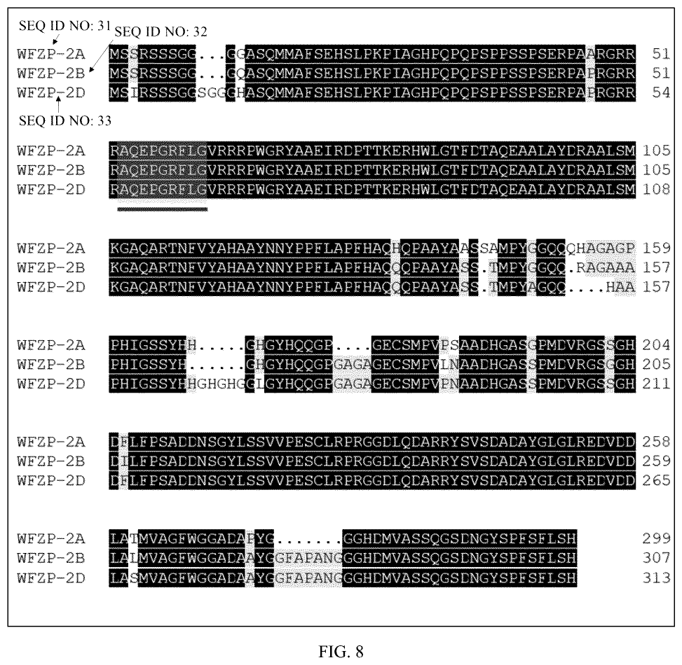

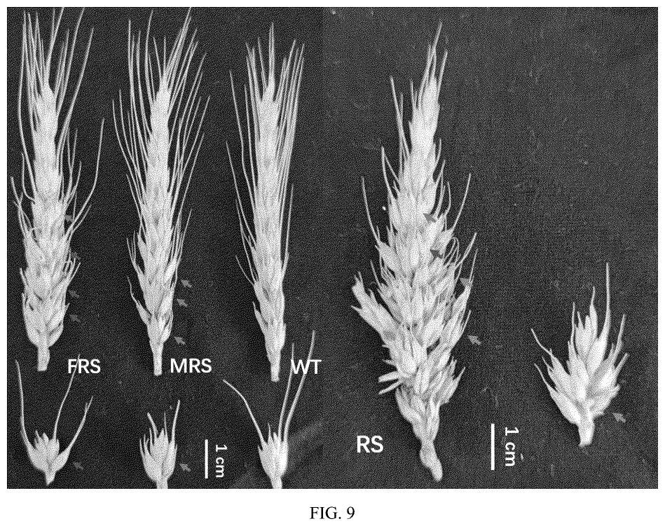

A clustered regularly interspersed short palindromic repeats (CRISPR-CAS9) technology is used, and the CRISPR-CAS9 tool stems from SINAGENE.INC. In the disclosure, conserved sequences of WFZP-2A, WFZP-2B and WFZP-2D of the hexaploid normal spike wheat variety “fielder” are edited, editing sites are shown in a sequence (GCAGGAGCCCGGGCGCTTCCTGG as shown in SEQ ID NO: 23) in the square frame of FIGS. 7A and 7B, and a corresponding protein sequence is shown in FIG. 8 (AQEPGRFLG as shown in SEQ ID NO: 24). Gene edited plants (as shown in FIG. 9) with RS, FRS, and MRS Are obtained through isolation, identification, and purification of gene edited positive plants, which indicates that the WFZP gene regulates the formation of the spike-branched (RS, FRS and MRS) of the wheat.

To sum up, in the disclosure, genetic analysis shows that the spike-branched trait of the tetraploid RS wheat GAN-A631 is jointly regulated by two pairs of recessive genes. It reveals that TtFZP-2A and TtFZP-2B genes located on Chr-2AS and Chr-2BS regulate the formation of RS, FRS, and MRS traits in the tetraploid wheat through the gene mapping of the spike-branched traits in the tetraploid wheat and the co-segregation analysis of the spike-branched traits with ttfzp-2A and ttfzp-2B gene variations. Gene editing of the hexaploid original spike wheat variety “fielder” reveals that the conserved protein regions of WFZP-2A, WFZP-2B, and WFZP-2D on Chr2 of the wheat regulate the formation of the spike-branched traits (RS, FRS, and MRS) of the wheat.

Finally, it should be noted that the above embodiments are merely used to illustrate the technical solution of the disclosure and not to limit it. Although the disclosure has been described in detail with reference to some embodiments, those skilled in the art should understand that the technical solution of the disclosure can be modified or equivalently replaced without departing from a purpose and a scope of the technical solution of the disclosure, which should be covered within the scope of claims of the disclosure.

What is claimed is:

1. Genes for regulating spike-branched traits of a wheat, comprising at least one of:
(1) TtFZP-2A and TtFZP-2B respectively located on Chr-2AS and Chr-2BS for regulating spike-branched traits of a tetraploid wheat; and
(2) WFZP-2A, WFZP-2B and WFZP-2D located on Chr 2 for regulating spike-branched traits of a hexaploid wheat.

2. Primer pairs, wherein the primer pairs are used for polymerase chain reaction (PCR) amplification of the genes for regulating the spike-branched traits of the wheat as claimed in claim 1.

3. The primer pairs as claimed in claim 2, comprising:
WFZP-AF with the nucleotide sequence shown in SEQ ID NO: 1, and WFZP-AR with the nucleotide sequence shown in SEQ ID NO: 2;
WFZP-BF with the nucleotide sequence shown in SEQ ID NO: 3, and WFZP-BR with the nucleotide sequence shown in SEQ ID NO: 4;
WFZP-DF with the nucleotide sequence shown in SEQ ID NO: 5, and WFZP-DR with the nucleotide sequence shown in SEQ ID NO: 6;
WFZP-BF2 with the nucleotide sequence shown in SEQ ID NO: 7, and WFZP-BR2 with the nucleotide sequence shown in SEQ ID NO: 8;
WFZP-AF2 with the nucleotide sequence shown in SEQ ID NO: 9, and WFZP-AR2 with the nucleotide sequence shown in SEQ ID NO: 10; and
WFZP-DF2 with the nucleotide sequence shown in SEQ ID NO: 11, and WFZP-DR2 with the nucleotide sequence shown in SEQ ID NO: 12.

4. A biomaterial, comprising the genes for regulating the spike-branched traits of the wheat as claimed in claim 1.

5. The biomaterial as claimed in claim 4, wherein the biomaterial comprises a gene expression cassette, engineered bacteria, or engineered cells.

6. An application method of the genes for regulating the spike-branched traits of the wheat as claimed in claim 1, comprising:
performing wheat genetic breeding by using the genes for regulating the spike-branched traits of the wheat.

7. An application method of the primer pairs as claimed in claim 2, comprising:
performing wheat genetic breeding by using the primer pairs.

8. An application method of the biomaterial as claimed in claim 4, comprising:
performing wheat genetic breeding by using the biomaterial.

9. The application method as claimed in claim 6, wherein in the tetraploid wheat, a plant with a homozygous single mutation of the TtFZP-2A exhibits a four-rowed spike trait, a plant with homozygous double mutations of the TtFZP-2A and the TtFZP-2B exhibits a ramified spike trait, and a plant with a homozygous mutation of the TtFZP-2A and a heterozygous mutation of the TtFZP-2B exhibits a multirow spike trait; and in the hexaploid wheat, a plant with mutations of the WFZP-2A, the WFZP-2B and the WFZP-2D exhibits the ramified spike trait, and a plant with a homozygous mutation of the WFZP-2A or the WFZP-2D exhibits the four-rowed spike trait.

10. A wheat germplasm resource with an edited wheat FRIZZY PANICLE (WFZP) gene, comprising: mutant genes leading to formation of a four-rowed spike trait and a ramified spike trait in wheat.


SEQUENCE LISTING (35 sequences)

SEQ ID NO: 1 - DNA, 25 bp, synthetic construct
  source 1..25: mol_type = other DNA
ctcacttcacttctgccatgagctc

SEQ ID NO: 2 - DNA, 24 bp, synthetic construct
  source 1..24: mol_type = other DNA
agcgcgtccgtttcagtgggagag

SEQ ID NO: 3 - DNA, 25 bp, synthetic construct
  source 1..25: mol_type = other DNA
gcatcacttcagttgtgccatgagc

SEQ ID NO: 4 - DNA, 25 bp, synthetic construct
  source 1..25: mol_type = other DNA
gccggtgcatttgcttcagtgtgag

SEQ ID NO: 5 - DNA, 25 bp, synthetic construct
  source 1..25: mol_type = other DNA
cacttcacttcagttctgccatgag

SEQ ID NO: 6 - DNA, 24 bp, synthetic construct
  source 1..24: mol_type = other DNA
gctggtgcatttgttgtttcagtg

SEQ ID NO: 7 - DNA, 22 bp, synthetic construct
  source 1..22: mol_type = other DNA
gcatcacttcagttgtgccatg

SEQ ID NO: 8 - DNA, 18 bp, synthetic construct
  source 1..18: mol_type = other DNA
caatgccggtgcatttgc

SEQ ID NO: 9 - DNA, 25 bp, synthetic construct
  source 1..25: mol_type = other DNA
ccacagtgctctcactccttctagc

SEQ ID NO: 10 - DNA, 21 bp, synthetic construct
  source 1..21: mol_type = other DNA
taagagcaatgccagcgcgtc

SEQ ID NO: 11 - DNA, 25 bp, synthetic construct
  source 1..25: mol_type = other DNA
ccacagtgctctcactcctagcttc

SEQ ID NO: 12 - DNA, 22 bp, synthetic construct
  source 1..22: mol_type = other DNA
ctgagagcaatgctggctggtg

SEQ ID NO: 13 - DNA, 25 bp, synthetic construct
  source 1..25: mol_type = other DNA
gcatcacttcagttgtgccatgagc

SEQ ID NO: 14 - DNA, 25 bp, synthetic construct
  source 1..25: mol_type = other DNA
gccggtgcatttgcttcagtgtgag

SEQ ID NO: 15 - DNA, 25 bp, synthetic construct
  source 1..25: mol_type = other DNA
ctcacttcacttctgccatgagctc

SEQ ID NO: 16 - DNA, 24 bp, synthetic construct
  source 1..24: mol_type = other DNA
agcgcgtccgtttcagtgggagag

SEQ ID NO: 17 - DNA, 23 bp, synthetic construct
  source 1..23: mol_type = other DNA
ccctctctcttcctctgtctaca

SEQ ID NO: 18 - DNA, 21 bp, synthetic construct
  source 1..21: mol_type = other DNA
atggcacaactgaagtgatgc

SEQ ID NO: 19 - DNA, 25 bp, synthetic construct
  source 1..25: mol_type = other DNA
ctcacttcacttctgccatgagctc

SEQ ID NO: 20 - DNA, 20 bp, synthetic construct
  source 1..20: mol_type = other DNA
ggtacgagctgccaatgtgc

SEQ ID NO: 21 - DNA, 19 bp, synthetic construct
  source 1..19: mol_type = other DNA
agcatctccaacaggcgtg

SEQ ID NO: 22 - DNA, 20 bp, synthetic construct
  source 1..20: mol_type = other DNA
atgtgtcttgtgagccgtgc

SEQ ID NO: 23 - DNA, 23 bp, synthetic construct
  source 1..23: mol_type = other DNA
gcaggagcccgggcgcttcctgg

SEQ ID NO: 24 - protein, 9 aa, synthetic construct
AQEPGRFLG

SEQ ID NO: 25 - DNA, 900 bp, synthetic construct
  source 1..900: mol_type = other DNA
atgagctctcgcagcagcagcggcggcggcggtgcctcccagatgatggccttctcggagcattcgctgccgaagccgatcgccggtcacccgcagccgcagccgtccccgccgtcgtcgccgagcgagcggccggcggcgcgcggcaggcggcgcgcgcaggagcccgggcgcttcctgggcgtgcgccggcggccgtggggccggtacgcggccgagatacgcgacccgaccaccaaggagcggcactggctcggcaccttcgacacggcgcaggaggccgccccggcctacgaccgcgccgcgctctccatgaagggcgcgcaggcgcgcaccaacttcgtctacgcgcacgccgcctacaacaactacccgcccttcctcgcgccgttccacgcgcagcaccagcccgccgcctacgccgcgtcctcggccatgccgtacggcggccagcagcagcacgcgggcgcggggccgccgcacattggcagctcgtaccaccacggccacggctaccaccagcagggcccgggcgagtgttccatgccggtgcccagtgccgcggatcacggcgccagcggcccgatggacgtgcgcggcagcagcggccacgacttcctcttccccagcgccgacgacaactccgggtacctgagcagcgtggtgccggagagctgcctccggccccgcggcggcgacctgcaggacgcgcggcgctactccgtgtccgacgccgacgcctacgggctgggcctccgggaggacgtggacgacctggcgacgatggtggccggcttctggggcggcgccgacgcgccgtacggcggcggccacgacatggtcgcctcgtcgcagggctcggacaacggctactcccccttcagcttcctctcccactga

SEQ ID NO: 26 - DNA, 900 bp, synthetic construct
  source 1..900: mol_type = other DNA
atgagctctcgcagcagcagcggcggcggcggtgcctcccagatgatggccttctcggagcattcgctgccgaagccgatcgccggtcacccgcagccgcagccgtccccgccgtcgtcgccgagcgagcggccggcggcgcgcggcaggcggcgcgcgcaggagcccgggcgcttcctgggcgtgcgccggcggccgtggggccggtacgcggccgagatacgcgacccgaccaccaaggagcggcactggctcggcaccttcgacacggcgcaggaggccgccctggcctacgaccgcgccgcgctctccatgaagggcgcgcaggcgcgcaccaacttcgtctacgcgcacgccgcctacaacaactacccgcccttcctcgcgccgttccacgcgcagcaccagcccgccgcctacgccgcgtcctcggccatgccgtacggcggccagcagcagcacgcgggcgcggggccgccgcacattggcagctcgtaccaccacggccacggctaccaccagcagggcccgggcgagtgttccatgccggtgcccagtgccgcggatcacggcgccagcggcccgatggacgtgcgcggcagcagcggccacgacttcctcttccccagcgccgacgacaactccgggtacctgagcagcgtggtgccggagagctgcctccggccccgcggcggcgacctgcaggacgcgcggcgctactccgtgtccgacgccgacgcctacgggctgggcctccgggaggacgtggacgacctggcgacgatggtggccggcttctggggcggcgccgacgcgccgtacggcggcggccacgacatggtcgcctcgtcgcagggctcggacaacggctactcccccttcagcttcctctcccactga

SEQ ID NO: 27 - DNA, 924 bp, synthetic construct
  source 1..924: mol_type = other DNA
atgagctctcgcagcagcagcggcggcggccaggcctcccagatgatggccttctcggagcattcgctgccgaagccgatcgccggccacccgcagccgcagccgtccccgccgtcgtcgccgagcgaacggccggcgcctcgcggcaggcggcgcgcgcaggagcccgggcgcttcctgggcgtgcgccggcggccgtggggccggtacgcggccgagatacgcgacccgaccaccaaggagcggcactggctcggcaccttcgacacggcgcaggaggccgccctggcctacgaccgtgccgcgctctccatgaagggcgcgcaggcgcgcaccaacttcgtctacgcgcacgccgcctacaacaactacccgcccttcctcgcgccgttccacgcgcagcagcagcccgccgcctacgcgtcctccaccatgccgtacggcgggcaacagcgcgcgggcgcggcggcgccgcacattggcagctcgtaccacggccacggctaccaccagcagggcccgggcgccggcgcgggcgagtgctccatgccggtgctcaatgccgcggatcacggcgccagcagcccgatggacgtgcgcggcagcggcggccacgacatcctcttccccagcgccgacgacaactccgggtacctgagcagcgtggtgccggagagctgcctccggccccgcggcggcgacctgcaggacgcgcggcgctactccgtgtccgacgccgacgcctacgggctgggcctccgggaggacgtggacgacctggcgttgatggtggccggcttctggggcggcgccgacgcggcgtacggcgggttcgcccccgcgaacggcggcggccacgacatggtcgcctcgtcgcagggctccgacaacggctactcccccttcagcttcctctcacactga

SEQ ID NO: 28 - DNA, 942 bp, synthetic construct
  source 1..942: mol_type = other DNA
atgagcatccgcagcagcagcggcggcagcggcggcggccatgcctcccagatgatggcgttctcggagcattcgctgccgaagccgatcgccggccacccgcagccgcagccgtccccgccgtcgtctccgagcgagaggccggcgccgcgtggcaggcggcgcgcgcaggagcccgggcgcttcctgggcgtgcgccggcggccgtggggccggtacgcggcggagatacgcgacccgaccaccaaggagcggcactggctcggcaccttcgacacggcgcaggaggccgccctggcctacgaccgcgccgcgctctccatgaagggcgcgcaggcgcgcaccaacttcgtctacgcgcacgccgcctacaacaactacccgcccttcctcgcgccgttccacgcgcagcagcagcccgccgcctacgcgtcctcgaccatgccgtacgccggccagcagcacgcggcgccgcacattggcagctcgtaccaccacggccacggccacggcggcctcggctaccaccagcagggcccgggcgccggcgcgggcgagtgctccatgccggtgcccaatgccgccgatcacggcgccagcagcccgatggacgtgcgcggcagcagcggccacgacttcctcttccccagcgccgacgacaactccgggtacctgagcagcgtggtgccggagagctgcctccggccccgcggcggcgacctgcaggacgcgcggcgctactccgtgtccgacgccgacgcctacgggctgggcctccgggaggacgtggacgacctggcgtccatggtggccggcttctggggcggcgccgacgcggcgtacggcgggttcgcccccgcgaacggcggcggccacgacatggtcgcctcgtcgcagggctccgacaacggctactcccccttcagcttcctctcccactga

SEQ ID NO: 29 - DNA, 924 bp, synthetic construct; identical to SEQ ID NO: 27
  source 1..924: mol_type = other DNA

SEQ ID NO: 30 - protein, 299 aa, synthetic construct
MSSRSSSGGGGASQMMAFSEHSLPKPIAGHPQPQPSPPSSPSERPAARGRRRAQEPGRFLGVRRRPWGRYAAEIRDPTTKERHWLGTFDTAQEAAPAYDRAALSMKGAQARTNFVYAHAAYNNYPPFLAPFHAQHQPAAYAASSAMPYGGQQQHAGAGPPHIGSSYHHGHGYHQQGPGECSMPVPSAADHGASGPMDVRGSSGHDFLFPSADDNSGYLSSVVPESCLRPRGGDLQDARRYSVSDADAYGLGLREDVDDLATMVAGFWGGADAPYGGGHDMVASSQGSDNGYSPFSFLSH

SEQ ID NO: 31 - protein, 299 aa, synthetic construct
MSSRSSSGGGGASQMMAFSEHSLPKPIAGHPQPQPSPPSSPSERPAARGRRRAQEPGRFLGVRRRPWGRYAAEIRDPTTKERHWLGTFDTAQEAALAYDRAALSMKGAQARTNFVYAHAAYNNYPPFLAPFHAQHQPAAYAASSAMPYGGQQQHAGAGPPHIGSSYHHGHGYHQQGPGECSMPVPSAADHGASGPMDVRGSSGHDFLFPSADDNSGYLSSVVPESCLRPRGGDLQDARRYSVSDADAYGLGLREDVDDLATMVAGFWGGADAPYGGGHDMVASSQGSDNGYSPFSFLSH

SEQ ID NO: 32 - protein, 307 aa, synthetic construct
MSSRSSSGGGQASQMMAFSEHSLPKPIAGHPQPQPSPPSSPSERPAPRGRRRAQEPGRFLGVRRRPWGRYAAEIRDPTTKERHWLGTFDTAQEAALAYDRAALSMKGAQARTNFVYAHAAYNNYPPFLAPFHAQQQPAAYASSTMPYGGQQRAGAAAPHIGSSYHGHGYHQQGPGAGAGECSMPVLNAADHGASSPMDVRGSGGHDILFPSADDNSGYLSSVVPESCLRPRGGDLQDARRYSVSDADAYGLGLREDVDDLALMVAGFWGGADAAYGGFAPANGGGHDMVASSQGSDNGYSPFSFLSH

SEQ ID NO: 33 - protein, 313 aa, synthetic construct
MSIRSSSGGSGGGHASQMMAFSEHSLPKPIAGHPQPQPSPPSSPSERPAPRGRRRAQEPGRFLGVRRRPWGRYAAEIRDPTTKERHWLGTFDTAQEAALAYDRAALSMKGAQARTNFVYAHAAYNNYPPFLAPFHAQQQPAAYASSTMPYAGQQHAAPHIGSSYHHGHGHGGLGYHQQGPGAGAGECSMPVPNAADHGASSPMDVRGSSGHDFLFPSADDNSGYLSSVVPESCLRPRGGDLQDARRYSVSDADAYGLGLREDVDDLASMVAGFWGGADAAYGGFAPANGGGHDMVASSQGSDNGYSPFSFLSH

SEQ ID NO: 34 - DNA, 2013 bp, synthetic construct
  source 1..2013: mol_type = other DNA
gtggttaatgttttagagcatcttcaacaggcgtcaaaaaatgcctcacgtgctaaaaaccatcgtttttgtgcgccggcgataaaaaaacagcgctccagcagatgctgtaaaatagagtacgcacaaaaaactttagcgcacgtgctaaaagcggtatcccagcctgcaaattctgggcgccggattatgcgcgctgcaaaatctgggctgctgcaggtggggccgccggattgggcgtcgtatttttgtgcatctggaaaacctcttcgtcgctcccgcgcacaaaaatactattttaacgctgtaaaagaattataccgtgccgaggcgaagcacgcctgttggagatgctcttagggaagacaaaggggcagctttctcctatgttcattctagtttgcacgtattgcgcaagcaagcacatggacacataagaaagcaaagccatatggtcagagtgtaaaaaagcacataattgtgtagcattttggcatgtatcataatagctattgtagtactagtatcttggagcatagattgcaattgtctctttcaaaacaaatagattacaattcttcaaaaggcttgatgcataggtaggtaagctctcctctttcccttcttttcctgtcagtggcaggcatctttacaccatcttattagtttgcaatacttggtactcagtagtaaatcatcaaagcatgcaaggcagtatgcccaactaacatagatgccacaacaagctatgcatgtaggaccattaataattaaagtgtttgtcttggcaggggcttcaatcggctctgccttcacaatcagtgtgtcatatactcatatgctggttgattaatttggagtggactagaatcatgacaaagatagtcatatctgaaactgacactaattaatacttaattgcaaggattgtagcatgcatgatattcataaatgaagtgtgcatgaaaactaagggcctgtttggttccaaataagtcaccaacttataagtcgaaaagtgaaaaaagtgacttattttgccaaacagacccgacttataagtcaccccaacttataagtcataagttgctccacccccaacttaaaacttataagtgaccctcttttgcgtgggtcccatcacctgcatgatgttgattggtgtggaaaggtgtgtcaggtgacttataagtcaggtgacaaccaaacatgcgtgacttataagtcactggttttaagtcaggtgacttattgaaaccaaacaggccctaagagtagaacattctaacttccaactgttatttttctagcaacacatacacacacatatatagctatatgtttgaccggcacaacattcccctcttctctttggtcctaaggttagggcacacaaatccaaacacaaggtgcatacatacaaactcgcaaacttggagtgagctttccattccctccttttactcctagaattaggccctgtttggttcataagtcctaggaccttttttagtcccaacttataagtctcaagtccctaaaaagtccctacctgtttggttcctgggacttataagtccatataagaccatattacaactataagtacctataagtccctccttgagagtcttatttcataagtcccaaacggccactttaagtccctataagttcctcctgtttggtttagatgggacttatagagacttttttaagtccctaagccaataaatccctggaaacaaacaccctcttaaactaaacaagagcaggtgcaatgcagcgcatgcatgtcactcccctcccctacccaaggtcgccatctcatccacagtgctctcagcctctcactccttctagcttctccctcccccgagctccccatttaaataccatcctgcctccctccatagtgagcactacacactagaagctcacagtctcagcaaccacctcctcaacctaagctagctagctcacacctcagagctcaagctaggcaggagcagt

SEQ ID NO: 35 - DNA, 2013 bp, synthetic construct
  source 1..2013: mol_type = other DNA
gtggttaatgttttagagcatctccaacaggcgtcaaaaaatgcctcacgtgctaaaaaccatcgtttttgtgcgccggcgataaaaaaacagcgctccagcagatgctgtaaaatagagtacgcacaaaaaactttagcgcacgcgctaaaagcggtatcccagcctgcaaattctgggcgccggattatgcgcgctgcaaaatctgggctgctgtaggtggggccgccggattgggcgccgtatttttgtgcatctggaaaacctattcatcgctcccgcgcacaaaaatactattttaacgctgtaaaagaattataccgtgccgaggcgaagcacgcctgttggagatgctcttagggaagacaaaggggcagctttctcctatgttcattctagtttgcacgtattgcgcaagcaagcacatggacacataagaaagcaaagccatatggtcagagtgtaaaaaagcacataattgtgtagcattttggcatgtatcataatagctattgtagtactagtatcttggagcatagattgcaattgtctctttcaaaacaaatagattacaattcttcaaaaggcttgatgcataggtaggtaagctctcctctttcccttcttttcctgtcagtggctggcatctttacaccatcttattagtttgcaatacttggtactcagtagtaaatcatcaaagcatgcaaggcagtatgcccaactaacatagatgccacaacaagctatgcatgtaggaccattaataattaaagtgtttgtcttggcaggggcttcaatcggctctgccttcacaatcagtgtgtcatatactcatatgctggttgattaatttggagtggactagaatcatgacaaagatagtcatatctgaaactgacactaattaatacttaattgcaaggattgtagcatgcatgatattcataaatgaagtgtgcatgaaaactaagggcctgtttggttccaaataagtcaccaacttataagtcgaaaaatgaaaaaagtgacttattttgccaaacagacccgacttataagtcaccccaacttataagtcataagttgttccacccccaacttaaaacttataagtgacccttttttgcgtgggtcccatcacctgcatgatgttgattggtgtggaaaggtgtgtcaggtgacttataagtcaggtgacaaccaaacatgcgtgacttataagtcactggttttaagtcaggtgacttattgaaaccaaacaggccctaagagtagaacattctaacttccaactgttatttttctagcaacacatacacacacatatatagctatatgtttgaccggcacaacattcccctcttctctttggtcctaaggttagggcacacaaatccaaacacaaggtgcatacatacaaactcgcaaacttggagtgagctttccattccctccttttactcctagaattaggccctgtttggttcataagtcctaggactttttttagtcccaacttataagtcccaagtccctaaaaagtccctacctgtttggttcctgggacttataagtccatataagaccatattacaactataagtacctataagtccctccttgagagtcttatttcataagtcccaaacggccactttaagtccctataagttcctcctgtttggtttagatgggacttatagagacttttttaagtccctaagccaataaatccctggaaacaaacaccctcttaaactaaacaagagcaggtgcaatgcagcgcatgcatgtcactcccctcccctacccaaggtcgccatctcatccacagtgctctcagcctctcactccttctagcttctccctcccccgagctccccatttaaataccatcctgcctccctccgtagtgagcactacacactagaagctcacagtctcagcaaccacctcctcaacctaagctagctagctcacacctcagagctcgagctagtcatcagcagt